(19) United States
(12) Patent Application Publication
(10) Pub. No.: US 2006/0084933 A1
(43) Pub. Date: Apr. 20, 2006

(54) Secret satisfying Stimulator

(76) Inventor: Robert Samuel Aellis, Newark, DE (US)

Correspondence Address: ROBERT S. AELLIS, 47 SHERIN DR, NEWARK, DE, 19702, US

(21) Appl. No.: 11/163,460

(22) Filed: Oct. 19, 2005

Related U.S. Application Data
(63) Continuation of application No. 60/522,608, filed on Oct. 20, 2004.

(51) Int. Cl. A61F 5/44

(52) U.S. Cl. 604/342

(57) ABSTRACT

A uniquely designed non automated device comprised of a piece of poly foam of a particular shape and size (as described in the illustrations) that is vinyl covered for easy cleaning and durability. It utilizes a metal ring that is attached to a 1″×36″ hook and loop fastening strap (for example Velcro). This strap and ring allow for the attachment to the foam piece of many various adult sexual aids readily available in the marketplace or those that the user may have already purchased. It does not include nor is it dependent upon any particular adult sexual aid. The drawings illustrate the attachment of the sexual aids. The sexual aid is placed through the metal ring. The fastening strap is then placed around the foam and looped through the opposite side of the ring. The strap is then adjusted and tightened after the sexual aid is positioned on top of the foam piece. The user is then able to straddle the foam piece and use the sexual device that has been placed and tightened allowing for ease of use. This significantly reduces stress to the body if the sexual aid was used without any anchoring device, as is the case in most present applications. Many sexual aids use suction cups to hold them in place such as the edge of a bathtub, the floor, a table etc. This prevents the user from being comfortable since there is no clearance from what it is attached to and increases the amount of physical stress to the body during use. The Secret Satisfying Stimulator solves that and can be placed anywhere, for example, on a bed, the floor, a chair, table etc. It is lightweight, and non-abrasive.

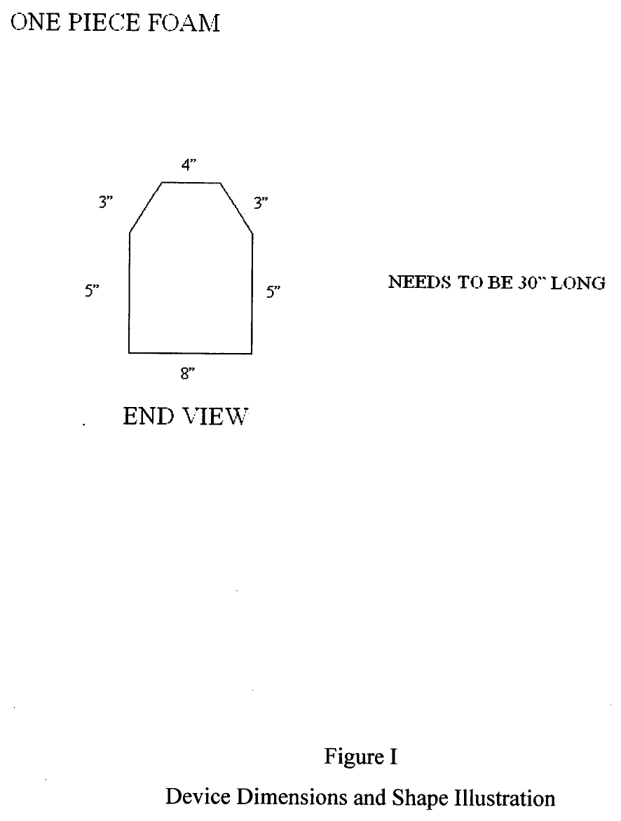

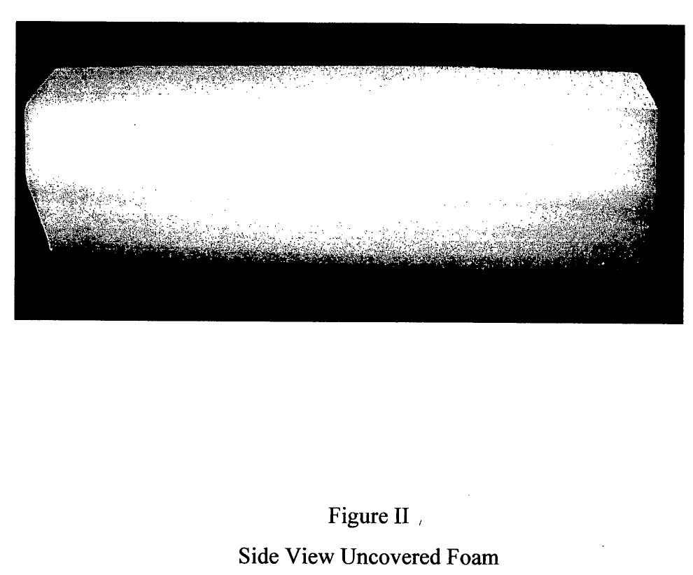

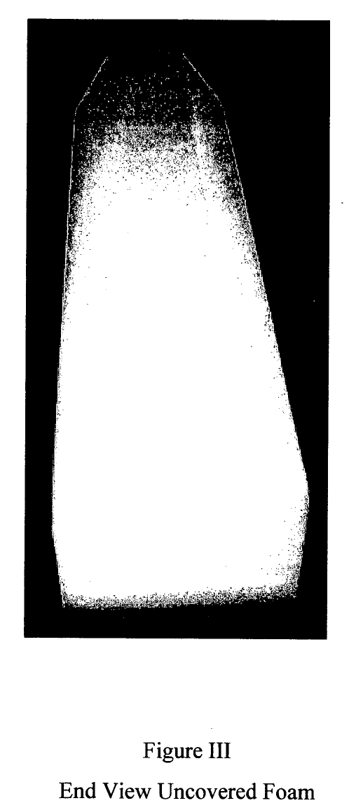

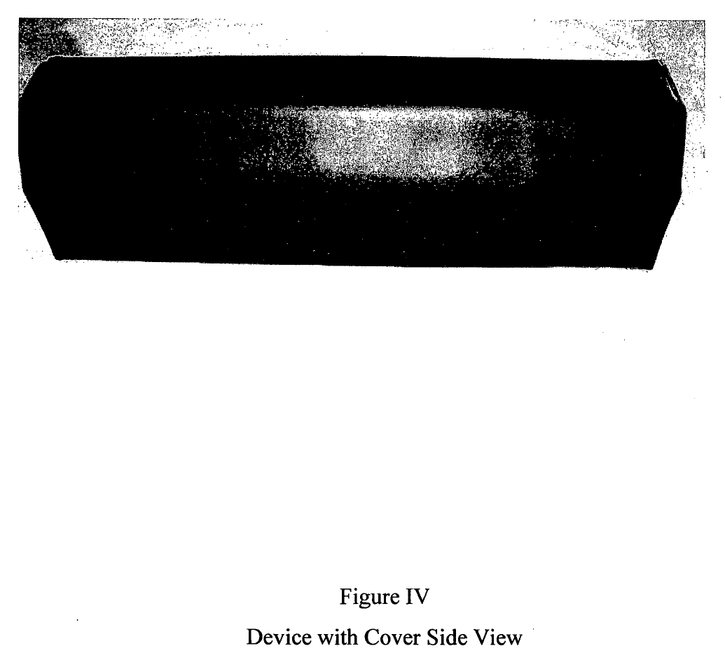

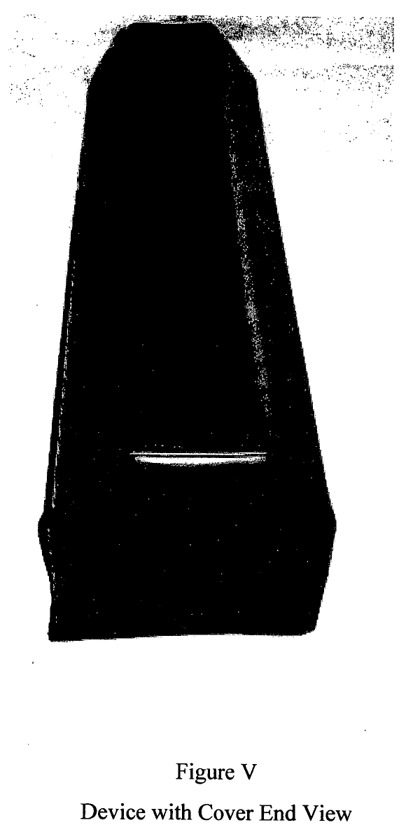

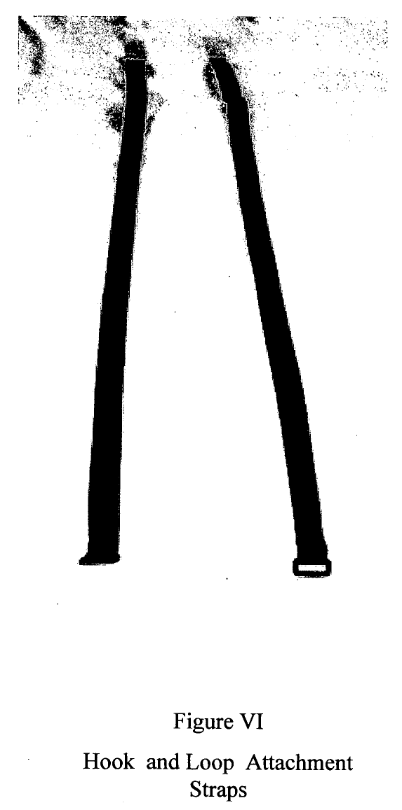

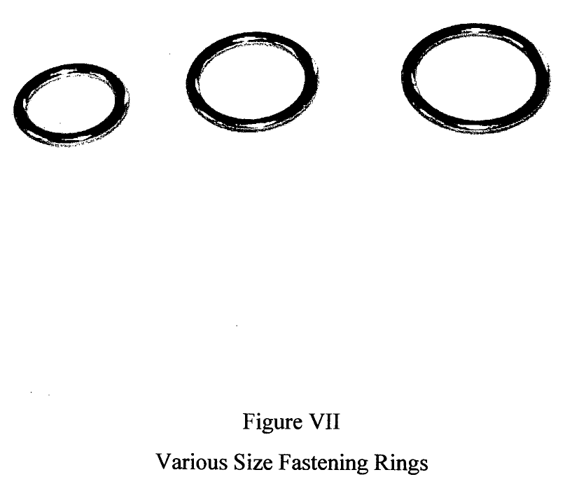

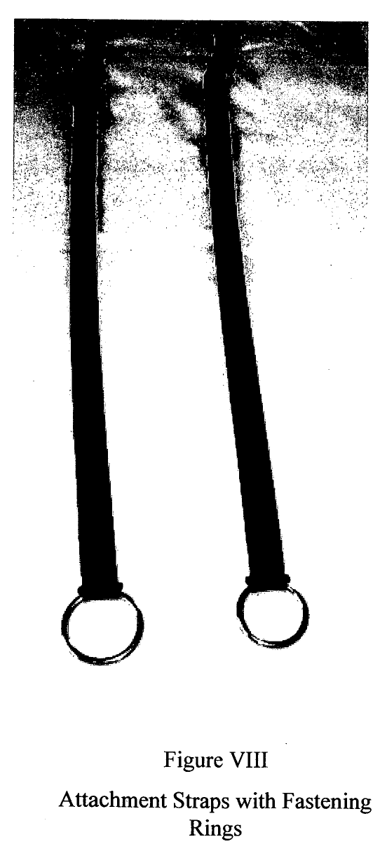

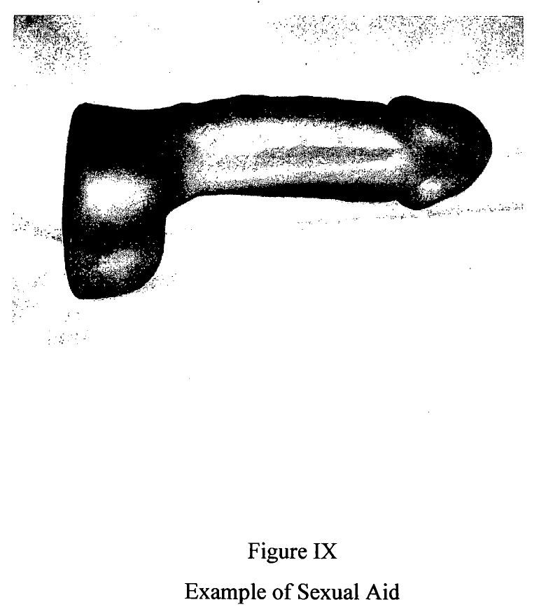

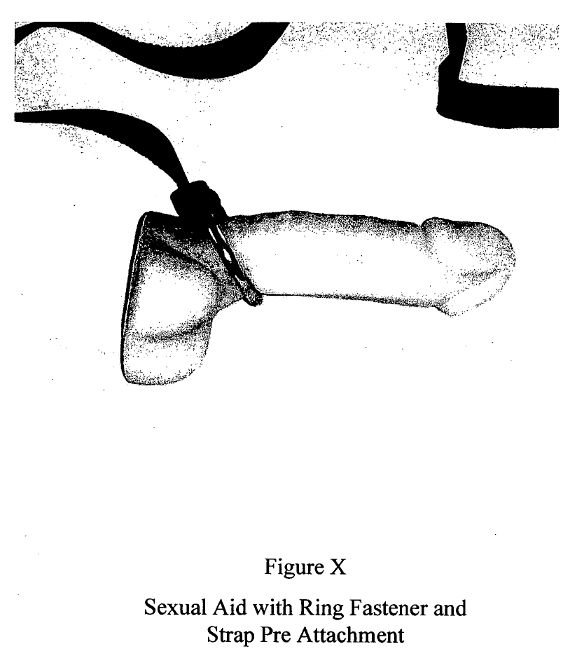

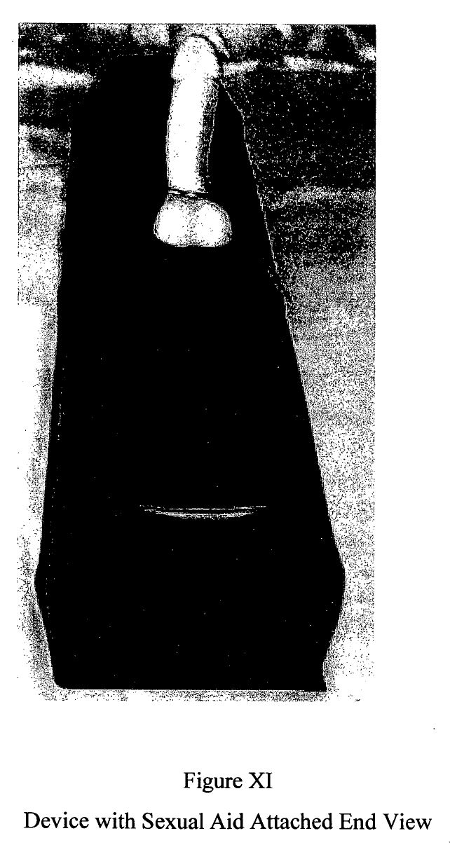

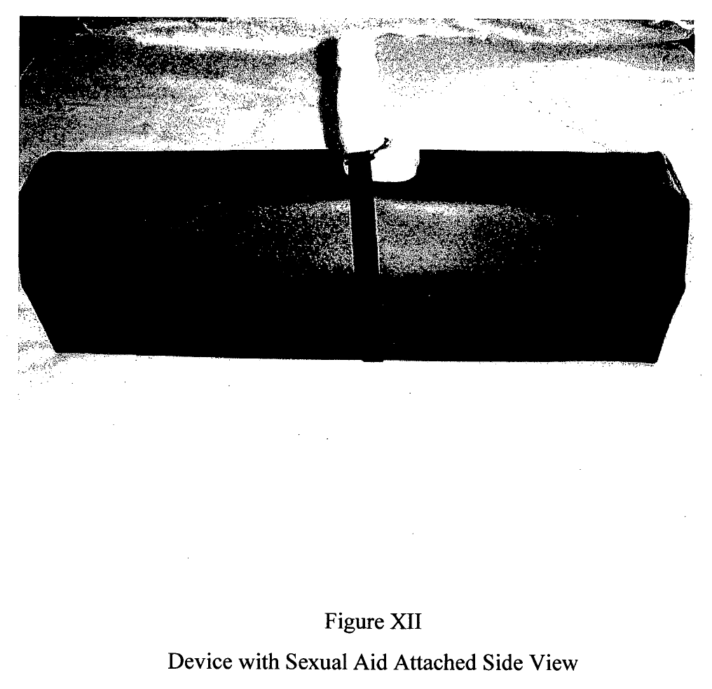

### BACKGROUND OF INVENTION

1. Field of Invention

This invention relates to male and female sexual aids specifically non-mechanized sexual aids.

2. Description of Prior Art

Modern life today presents many new problems to human sexuality. First and foremost we are faced with a plague of unparalleled proportions known as AIDS. In addition there are many sexually transmitted viral and parasitic diseases which until recently were not widely recognized. Diseases such as Herpes II, genital warts, trichomoniasis, chlamydia, etc. Bacterial diseases like Syphilis and Gonorrhea. The current economic downturn and multiple income families have all had an adverse effect on male and female sexuality. If men and women do not receive frequent enough sexual satisfaction it can lead to behavioral problems. These certainly include practicing unsafe sex, infidelity and more. It is the aim of the present invention to substantially improve male and female sexual satisfaction.

Dildoes, vibrators, anal plugs etc already exist in many sizes and styles. They can be purchased at adult book and novelty stores, home parties, and the Internet. Many of these products sell for $150.00 or less. A premium female mastubatory device for women made by Abeo Research of Monticello, Illinois retails for about $1,400.00. It is called the Sybian. The present invention is also a premium product. It allows the user to accurately simulate intercourse. It functions hands off and contains no motors or other automated devices. This product is designed to help satisfy male and female sexual urges and has a general therapeutic use. Its design allows for initiation primarily to occur from above. Men and women in general and those suffering from impotence either circulatory or physiological in origin will benefit.

U.S. Patent No. 43,910,262 dated Oct. 7, 1975 describes the principal prior art by inventor Dan T. Stoughton. This is a therapeutic apparatus intended for producing orgasms in males and females. It contained a motor driven hydraulic which produce positive and negative pneumatic pulses.

### SUMMARY

The present invention contains no moving parts or motors and is designed to help produce orgasms in men and women. Its unique shape has been developed to reduce physical body stress and utilize many adult products readily available to men and women. It will allow many of these products that the consumer may have already purchased to be used. Its goal is to assist men and women achieve orgasm at a pace that can be controlled by them. It is very simple, easy to use, easy to clean and sanitize, very discreet. It is priced reasonably and is designed to help consumers use their existing sexual aids. It is not dependent on any other mechanized or non-mechanized device in order to use.

### BRIEF DESCRIPTION OF DRAWINGS—REVISED

FIG. I Device Dimensions and Shape Illustration

FIG. II Side View of Uncovered Foam

FIG. III End View of Uncovered Foam

FIG. IV Device with Cover Side View

FIG. V Device with Cover End View

FIG. VI Hook and Loop Attachment Straps

FIG. VII Various Size Fastening Rings

FIG. VIII Attachment Straps with Fastening Rings

FIG. IX Example of Sexual Aid

FIG. X Sexual Aid with Ring and Strap Pre Attachment

FIG. XI Device with Sexual Aid Attached End View

FIG. XII Device with Sexual Aid Attached Side View

What is claimed is:

1. The embodiments of the invention in which an exclusive property or privilege exists are claimed and defined as:
1. A. masturbatory device comprising
B. A premium vinyl covered piece of poly foam of a uniquely developed shape and size that is washable and sanitizable
C. A means for attaching adult sexual aids via the use of a metallic ring and a hook and loop fastener
D. Foam and covering is of a particular shape as illustrated in the drawings.

2. The Masturbatory device of claim wherein: said means for providing a comfortable means for the user to straddle and control their motion.
